(19) United States
(12) Patent Application Publication
(10) Pub. No.: US 2015/0133789 A1
(43) Pub. Date: May 14, 2015

(54) PUNCTURE APPARATUS

(71) Applicant: TERUMO KABUSHIKI KAISHA, Shibuya-ku (JP)

(72) Inventors: Shigeki ARIURA, Ebina-shi (JP); Masakatsu Kawaura, Sunnyvale, CA (US)

(73) Assignee: TERUMO KABUSHIKI KAISHA, Shibuya-ku (JP)

(21) Appl. No.: 14/600,278

(22) Filed: Jan. 20, 2015

Related U.S. Application Data
(63) Continuation of application No. PCT/JP2012/068356, filed on Jul. 19, 2012.

(51) Int. Cl. A61B 17/34; A61B 8/14; A61B 8/08

(52) U.S. Cl. CPC A61B 17/3403; A61B 17/3494; A61B 17/3468; A61B 8/0841; A61B 8/14; A61B 2017/3413; A61F 2/0045
USPC 600/461

(57) ABSTRACT

A puncture apparatus is disclosed, which can include an elongated insertion portion configured to be inserted into a living body lumen, a puncture needle configured to puncture a living body tissue near the insertion portion from a part different from the living body lumen, with the insertion portion inserted in the living body lumen, a puncture needle guide portion, which movably supports the puncture needle and which determines a puncture path for a needle tip of the puncture needle when the puncture needle punctures the living body tissue, an ultrasonic transmission-reception section in the insertion portion or the puncture needle guide portion, for transmission and reception of an ultrasonic wave, and an image data production section for producing image data for displaying on a display section an image including the living body lumen and a predicted path of the puncture path, based on information acquired by the ultrasonic transmission-reception section.

### CROSS-REFERENCES TO RELATED APPLICATIONS

This application is a continuation of International Application No. PCT/JP2012/068356 filed on Jul. 19, 2012, the entire content of which is incorporated herein by reference.

### TECHNICAL FIELD

The present disclosure generally relates to a puncture apparatus.

### BACKGROUND

If a person suffers from a urinary incontinence, for example, if a person suffers from a stress urinary incontinence, then urine leakage can be caused by application of abdominal pressure during normal exercise or by laughing, coughing, sneezing, or the like. The cause of this may be, for example, that the pelvic floor muscle which is a muscle for supporting the urethra is loosened by birth.

For the treatment of urinary incontinence, a surgical treatment is effective, in which there is used, for example, a tape-shaped implant called a “sling.” The sling is indwelled inside the body and the urethra is supported by the sling (see, for example, Japanese Patent Laid-Open No. 2010-99499). In order to indwell the sling inside the body, an operator would incise the vagina with a surgical knife, dissect a region between the urethra and the vagina, and make the dissected region and the outside communicate with each other through an obturator foramen by use of a puncture needle, forming a puncture hole. The formation of the puncture hole by use of the puncture needle is conducted by a blind operation, depending only on the feeling to the operator's fingers. Then, by use of such a puncture hole, the sling is indwelled into the body.

In the method of indwelling a sling by use of a conventional medical instrument such as a surgical knife, however, the procedure can be invasive and the burdensome on the patient. In addition, at the time of incising the vagina with a surgical knife and dissecting the region between the urethra and the vagina, an error in the dissecting direction may be made, leading to a risk of indwelling the sling in an improper position or a risk of damaging the urethra. Further, there can be a fear that the operator might damage his/her fingertip. In some cases, furthermore, a patient's movement or the like during the formation of the puncture hole by the puncture needle may cause a deviation of a puncture path of the puncture needle from an intended path, which problem may be left unnoticed. If unnoticed, this may result in that the urethra or the like is damaged, that the sling cannot be indwelled into a proper position, or that there will occur complications such as the exposure of the sling into the vagina by breaking through the vaginal wall after the operation, a phenomenon called “erosion.”

### SUMMARY

In accordance with an exemplary embodiment, a puncture apparatus is disclosed that is able to avoid puncturing a part of the urethra and/or vaginal wall which should not be punctured, at the time of puncturing a living body tissue with a puncture needle, and also able to reduce the relative burden on the patient with a relatively high degree of safety to both the patient and the operator.

In accordance with an exemplary embodiment, a puncture apparatus is disclosed, which can include an elongated insertion portion configured to be inserted into a living body lumen. The puncture apparatus can also include a puncture needle configured to puncture a living body tissue near the insertion portion from a part different from the living body lumen, with the insertion portion inserted in the living body lumen. The puncture apparatus can include a puncture needle guide portion, which movably supports the puncture needle and determines a puncture path for a needle tip of the puncture needle when the puncture needle punctures the living body tissue. The puncture apparatus can include an ultrasonic transmission-reception section, which is provided in the insertion portion or the puncture needle guide portion, for transmission and reception of an ultrasonic wave. The puncture apparatus can further include an image data production section for producing image data for displaying on a display section an image including the living body lumen and a predicted path of the puncture path, based on information acquired by the ultrasonic transmission-reception section.

In accordance with an exemplary embodiment, the puncture apparatus further includes a puncture path changing section for changing the puncture path.

In accordance with an exemplary embodiment, the puncture path changing section preferably changes a position of the puncture needle relative to the insertion portion.

In accordance with an exemplary embodiment, the puncture further includes a detection section for detecting a positional relationship between the insertion portion and the puncture needle, wherein the image data production section is configured to determine the predicated path of the puncture path, based on results of detection by the detection section.

In accordance with an exemplary embodiment, wherein in a case where the predicted path of the puncture path is inappropriate, the puncture path is preferably changed by the puncture path changing section so that the predicated path will be appropriate.

In accordance with an exemplary embodiment, the puncture needle has a bent region, and is rotatably disposed in the puncture needle guide portion. For example, the puncture needle guide portion can be configured to determine the puncture path such that a needle tip of the puncture needle passes a far-side or a near-side from a rotation center of the puncture needle compared with the insertion portion when the puncture needle moves rotationally and punctures the living body tissue.

In accordance with an exemplary embodiment, the puncture needle guide portion is preferably configured to determine the puncture path such that the needle tip of the puncture needle passes a vicinity of the insertion portion while avoiding the insertion portion.

In accordance with an exemplary embodiment, the living body lumen is a urethra or a vagina.

In accordance with an exemplary embodiment, the puncture apparatus includes an elongated first insertion portion configured to be inserted into a first living body lumen. The puncture apparatus can also include an elongated second insertion portion configured to be inserted into a second living body lumen arranged side by side with the first living body lumen. The puncture apparatus can include a puncture needle configured to puncture a living body tissue near the first insertion portion and the second insertion portion in a state where the first insertion portion is inserted in the first living body lumen and the second insertion portion is inserted in the second living body lumen. The puncture apparatus can include a puncture needle guide portion which movably supports the puncture needle and which determines a puncture path for a needle tip of the puncture needle when the puncture needle punctures the living body tissue. The puncture apparatus can also include an ultrasonic transmission-reception section provided in the first insertion portion, the second insertion portion, or the puncture needle guide portion, for transmitting and receiving an ultrasonic wave. The puncture apparatus can further include an image data production section for producing image data for displaying on a display section an image including the first living body lumen, the second living body lumen, and a predicted path of the puncture path, based on information acquired by the ultrasonic transmission-reception section.

In accordance with an exemplary embodiment, the puncture needle preferably punctures the living body tissue from a part different from the first living body lumen and the second living body lumen.

In accordance with an exemplary embodiment, the puncture needle has a bent region, and is rotatably disposed in the puncture needle guide portion. For example, the puncture needle guide portion can be configured to determine the puncture path such that the needle tip of the puncture needle passes between the first insertion portion and the second insertion portion when the puncture needle moves rotationally and punctures the living body tissue.

In accordance with an exemplary embodiment, a puncture apparatus is disclosed, which can include an elongated first insertion portion configured to be inserted into a first living body lumen, of the first living body lumen and a second living body lumen which are arranged side by side. The puncture apparatus also includes a puncture needle configured to puncture a living body tissue, with the first insertion portion inserted in the first living body lumen. The puncture apparatus also includes a puncture needle guide portion which rotatably supports the puncture needle and which determines a puncture path for a needle tip of the puncture needle such that the needle tip of the puncture needle passes a vicinity of the first insertion portion while avoiding the first insertion portion when the puncture needle punctures the living body tissue. The puncture apparatus also includes an ultrasonic transmission-reception section provided in the first insertion portion or the puncture needle guide portion and performs, for transmitting and receiving an ultrasonic wave. The puncture apparatus further includes an image data production section for producing image data for displaying on a display section an image including the first living body lumen, the second living body lumen, and a predicted path of the puncture path, based on information acquired by the ultrasonic transmission-reception section.

In accordance with an exemplary embodiment, the puncture apparatus preferably further includes an elongated second insertion portion configured to be inserted into the second living body lumen, wherein the ultrasonic transmission-reception section is provided in the first insertion portion, the second insertion portion, or the puncture needle guide portion, and the puncture needle is configured to puncture the living body tissue in a state in which the first insertion portion is inserted in the first living body lumen and the second insertion portion is inserted in the second living body lumen.

In accordance with an exemplary embodiment, the puncture needle is preferably configured to puncture the living body tissue from the second living body lumen.

### BRIEF DESCRIPTION OF THE DRAWINGS

FIGS. 1A and 1B are views showing a first exemplary embodiment of a puncture apparatus according to the present disclosure;

FIG. 2 is a side view showing a state in which members are removed from the puncture apparatus shown in FIGS. 1A and 1B;

FIG. 3 is a block diagram of the puncture apparatus shown in FIGS. 1A and 1B;

FIGS. 4A to 4C are views showing an operating procedure of the puncture apparatus shown in FIGS. 1A and 1B in accordance with an exemplary embodiment;

FIGS. 5A and 5B are views showing an operating procedure of the puncture apparatus shown in FIGS. 1A and 1B in accordance with an exemplary embodiment;

FIGS. 6A and 6B are views showing an operating procedure of the puncture apparatus shown in FIGS. 1A and 1B in accordance with an exemplary embodiment;

FIGS. 7A and 7B are views showing an operating procedure of the puncture apparatus shown in FIGS. 1A and 1B in accordance with an exemplary embodiment;

FIGS. 8A and 8B are views showing an operating procedure of the puncture apparatus shown in FIGS. 1A and 1B in accordance with an exemplary embodiment;

FIG. 9 is a view showing an operating procedure of the puncture apparatus shown in FIGS. 1A and 1B in accordance with an exemplary embodiment;

FIG. 10 is a view illustrating a second exemplary embodiment of the puncture apparatus according to the present disclosure;

FIGS. 11A to 11F are views illustrating a third exemplary embodiment of the puncture apparatus according to the present disclosure;

FIGS. 12A and 12B are views showing an operating procedure of the puncture apparatus shown in FIGS. 11A to 11F in accordance with an exemplary embodiment;

FIGS. 13A and 13B are views showing an operating procedure of the puncture apparatus shown in FIGS. 11A to 11F in accordance with an exemplary embodiment; and

FIGS. 14A to 14F are views illustrating a fourth exemplary embodiment of the puncture apparatus according to the present disclosure.

### DETAILED DESCRIPTION

Now, the puncture apparatus of the present disclosure will be described in detail below, referring to exemplary embodiments shown in the accompanying drawings.

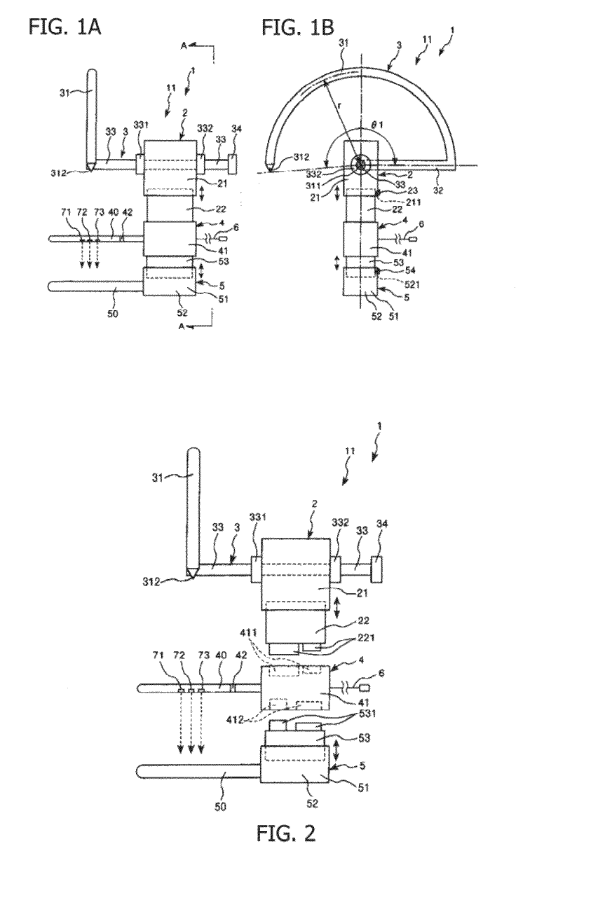

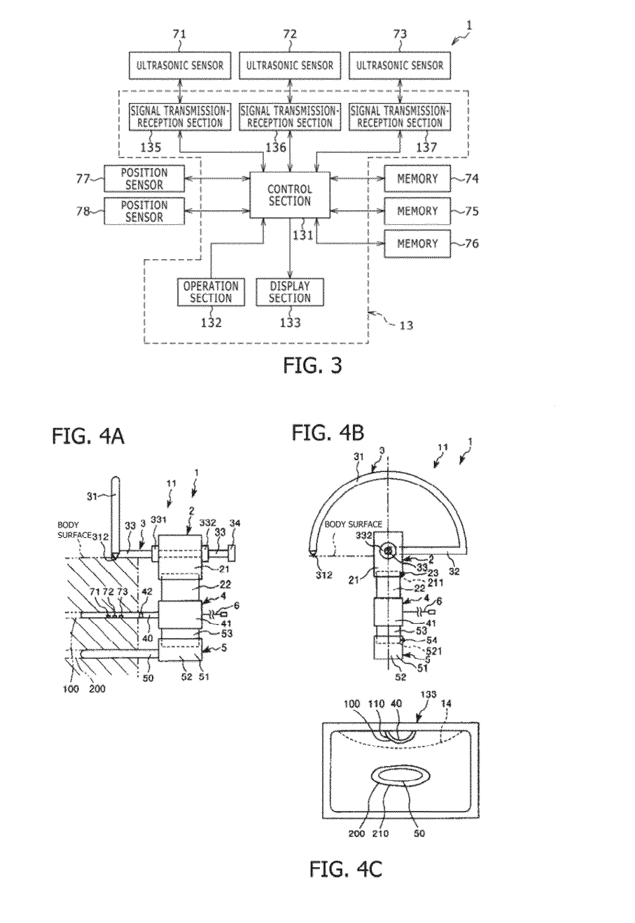

FIGS. 1A and 1B are views showing a first exemplary embodiment of the puncture apparatus of the present disclosure, wherein FIG. 1A is a side view and FIG. 1B is a sectional view taken along line A-A in FIG. 1A. FIG. 2 is a side view illustrating a state where members are removed from the puncture apparatus shown in FIGS. 1A and 1B. FIG. 3 is a block diagram of the puncture apparatus shown in FIGS. 1A and 1B. FIGS. 4A to 9 are views showing an operating procedure of the puncture apparatus shown in FIGS. 1A and 1B in accordance with an exemplary embodiment.

FIG. 4A is a side view. In addition, FIGS. 4B, 5A, 6A, 7A, 8A, and 9 are sectional views corresponding to the sectional view taken along line A-A in FIG. 1A. Besides, FIGS. 4C, 5B, 6B, 7B, and 8B are view illustrating an image displayed on a display section.

In addition, in FIGS. 4B, 5A to 8A, and 9, that portion of a puncture member which is puncturing a living body tissue and therefore invisible in itself is shown, for easy understanding. In FIGS. 4B, 5A to 8A, and 9, male screws are shown schematically. Further, in FIGS. 4C, and 5B to 8B, the same symbols as used for real parts are used in an image displayed on the display section.

In the following description, the left side in FIGS. 1A, 2, and 4A will be referred to as the “distal end,” the right side in the figures as the “proximal end,” the upper side in FIGS. 1A, 1B, 2, 4B, and 5A to 8A as the “upper side,” the lower side in the figures as the “lower side,” the upper side in FIGS. 4C and 5B to 8B as the “upper side,” the lower side in the figures as the “lower side,” the right side in the figures as the “right side,” and the left side in the figures as the “left side.”

In accordance with an exemplary embodiment, the puncture apparatus 1 shown in FIGS. 1-14F is an apparatus that can be used in treatment of female urinary incontinence, for example, to be used at the time of implanting into a living body an implant (in-living-body indwelling instrument) for treatment of urinary incontinence.

In accordance with an exemplary embodiment, the implant can be an instrument, which is implantable for treatment of female urinary incontinence. For example, the implant is an instrument, which is implanted, in a living body to support a urethra, for example, an instrument, which supports the urethra in the manner of pulling the urethra in a direction for spacing away from vaginal wall when the urethra would otherwise be going to move toward the vaginal wall. For example, a flexible elongated member can be used as the implant.

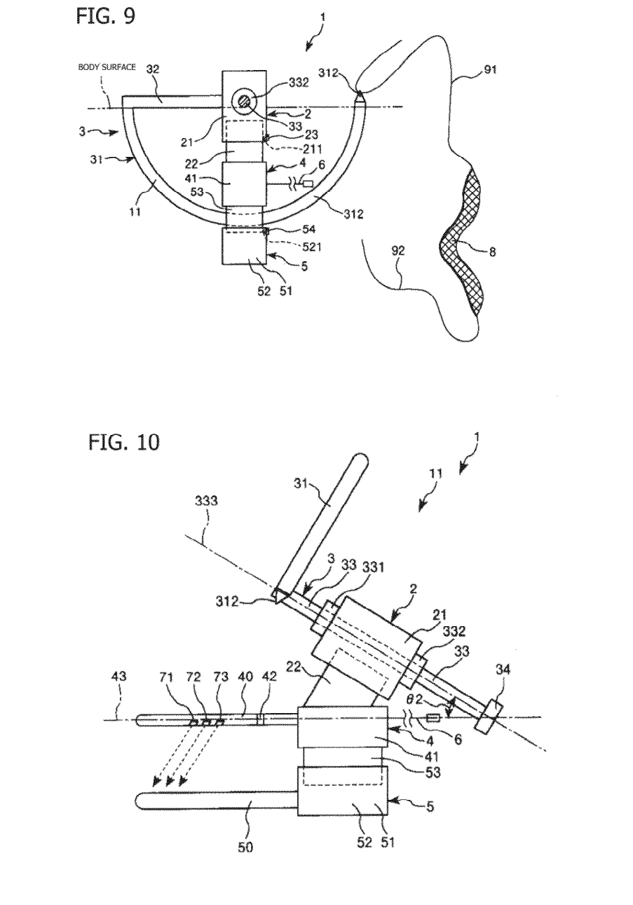

As shown in FIG. 9, an implant 8 in this exemplary embodiment can be net-like in form and can be band-like (ribbon-like) in entire shape. The implant 8 is called a “sling.” The implant 8 may be composed of a body knitted in a net form (lattice form) by intersection of filamentous elements, for example, composed of a net-like knitting. Examples of the filamentous elements can include those which are circular in cross-sectional shape, and those which are flat in cross-sectional shape, for example, which are band-like (ribbon-like) in shape. A one-side end of a string 91 is fixed to one-side end of the implant 8, and a one-side end of a string 92 is fixed to the other-side end of the implant 8.

The material constituting the implant 8 is not specifically restricted; examples of the material usable here can include various biocompatible resin materials (such as polypropylene), their fibers and the like.

The material or materials constituting the strings 91 and 92 are not specifically restricted; examples of the material or materials applicable here can include various biocompatible resin materials (such as polypropylene), their fibers and the like.

In addition, the implant 8 is not restricted to the net-like ones.

As shown in FIGS. 1A to 3, a puncture apparatus 1 can include a puncture apparatus main body 11, and a control and display unit 13 electrically connected to the puncture apparatus main body 11 via a cable 6.

As shown in FIGS. 1A, 1B, and 2, the puncture apparatus main body 11 can include a urethral-insertion member 4 having an elongated urethral-insertion portion (first insertion portion) 40 which is inserted into a urethra (first living body lumen), a vaginal-insertion member 5 having an elongated vaginal-insertion portion (second insertion portion) which is inserted into a vagina (second living body lumen), a puncture member 3 having a puncture needle 31 which punctures a living body tissue in a state in which the urethral-insertion portion 40 is inserted in the urethra and the vaginal-insertion portion 50 is inserted in the vagina, and a puncture needle guide member 2 having a puncture needle guide portion 21 which rotatably holds (supports) the puncture member 3 and which determines a puncture path for a needle tip of the puncture needle 31 when the puncture needle 31 punctures the living body tissue. The puncture member 3 can include the puncture needle 31, which punctures a living body tissue, an axial portion 33, and an interlock portion 32, which interlocks the puncture needle 31, and the axial portion 33. The puncture member 3 can be so configured as to puncture a living body tissue in the vicinity of the urethra and the vagina via a part different from the urethra and the vagina.

The puncture needle guide member 2, the urethral-insertion member 4, and the vaginal-insertion member 5 are arranged in this order from the upper side toward the lower side in FIGS. 1A and 1B. In addition, the puncture needle guide member 2 can be attached to the urethral-insertion member 4 in a freely detachable manner. In accordance with an exemplary embodiment, the vaginal-insertion member 5 can be attached to the urethral-insertion member 4 in a freely detachable manner. The puncture apparatus 1 is used in its assembled state as shown in FIGS. 1A and 1B.

The puncture needle guide member 2 can include the puncture needle guide portion 21, and a movable portion 22 disposed to be movable vertically in FIGS. 1A and 1B relative to the puncture needle guide portion 21. In accordance with an exemplary embodiment, an upper end portion of the movable portion 22 is inserted inside a lower end portion of the puncture needle guide portion 21. In addition, the puncture member 3 can be rotatably held by the puncture needle guide portion 21, whereby the puncture needle 31 is movably held (supported) by the puncture needle guide portion 21.

In accordance with an exemplary embodiment, the puncture needle guide member 2 can have a male screw 23, and a wall portion at the lower end portion of the puncture needle guide portion 21 can be formed with a female screw 211 for engagement with the male screw 23. When the male screw 23 is rotated in a predetermined direction, a tip of the male screw 23 can be pressed against the movable portion 22, thereby inhibiting the movable portion 22 from moving relative to the puncture needle guide portion 21. When the male screw 23 is rotated in a direction reverse to the above, the tip of the male screw 23 can be separated from the movable portion 22, permitting the movable portion 22 to move relative to the puncture needle guide portion 21. The rotation of the male screw 23 can help enable an adjustment of the separated distances between the puncture needle guide portion 21 and the urethral-insertion portion 40 and the vaginal-insertion portion 50, for example, the separated distances between the axial portion 33 of the puncture member 3 and the urethral-insertion portion 40 and the vaginal-insertion portion 50. In accordance with an exemplary embodiment, the position of the puncture needle 31 can be changed relative to the urethral-insertion portion 40 and the vaginal-insertion portion 50.

In accordance with an exemplary embodiment, the male screw 23 and the female screw 211 can constitute a lock portion for change-over between a state in which the movable portion 22 is movable relative to the puncture needle guide portion 21 and a state in which such movement of the movable portion 22 is inhibited. In addition, the puncture needle guide portion 21, the movable portion 22, the male screw 23, and the female screw 211 can constitute a puncture path changing section for changing the puncture path.

In accordance with an exemplary embodiment, a detection section for detecting a positional relationship between the urethral-insertion portion 40 and the puncture needle 31, a position sensor 77 for detecting the position of the movable portion 22 relative to the puncture needle guide portion 21 is disposed inside the puncture needle guide member 2 (see FIG. 3). As the position sensor 77, there can be used, for example, one whose electric resistance varies according to the position of the movable portion 22 relative to the puncture needle guide portion 21. The results of detection by the position sensor 77 can be transmitted through the cable 6 to a control section 131 in the control and display unit, in a state where a connector 221 (described later) and a slot 411 (described later) are connected to each other. Based on the detection results, the control section 131 can determine the separated distance between the urethral-insertion portion 40 and the puncture needle guide portion 21.

In addition, a storage section for storing various kinds of information, a memory 74 can be disposed inside the movable portion 22 (see FIG. 3).

In accordance with an exemplary embodiment, the connector 221 electrically connected to the position sensor 77 and the memory 74 can be provided at a lower end portion of the movable portion 22.

In addition, intrinsic information of the puncture member 3, for example, can be stored in the memory 74. Examples of the intrinsic information on the puncture member 3 can include a radius r of an arc of the puncture needle 31 of the puncture member 3 (see FIG. 1B), and an index indicating whether the puncture member 3 is an unused one or not. The index may be information indicating whether or not the connector 221 and the slot 411 (described later) of the urethral-insertion member 4 have been connected to each other at least once. The various kinds of information stored in the memory 74 can be transmitted through the cable 6 to the control section 131 in the control and display unit when the connector 221 and the slot 411 are connected to each other. When there is no history of connection between the connector 221 and the slot 411, the control section 131 can determines that the puncture member 3 is an unused puncture member 3. When there is a history of at least one-time connection, the control section 131 determines that the puncture member 3 is a used puncture member 3.

The urethral-insertion member 4 can include the urethral-insertion portion 40, and a supporting portion 41, which supports the urethral-insertion portion 40. In this exemplary embodiment, the urethral-insertion portion 40 is firmly attached to the supporting portion 41.

The urethral-insertion portion 40 can be formed of a non-flexible rigid material, and can be straight, bar-like in shape. In addition, a distal portion of the urethral insertion portion 40 can be rounded, which helps permits smooth insertion of the urethral-insertion portion 40 into the urethra.

In accordance with an exemplary embodiment, the urethral-insertion portion 40 may be tubular-shaped. Where the urethral-insertion portion 40 is tubular, for example, a balloon catheter can be used in the state of being inserted in the urethral-insertion portion 40. In that case, by inserting the balloon into a bladder, the balloon can be used as a position restriction section for restricting the position of the urethral-insertion portion 40 in the longitudinal direction.

In addition, the urethral-insertion portion 40 can include a marker 42 on an outer circumferential portion of the urethral-insertion portion 40. The marker 42 can be so disposed that the marker 42 is located at a urethral orifice when the urethral-insertion portion 40 is inserted in the urethra and a distal portion of the urethral-insertion portion 40 is located directly in front of the bladder.

In accordance with an exemplary embodiment, the urethral-insertion portion 40 can be provided with three ultrasonic sensors (ultrasonic transducers) 71, 72, and 73 as ultrasonic transmission-reception sections for transmission and reception of ultrasonic waves. Each of the ultrasonic sensors 71, 72, and 73 transmits an ultrasonic wave toward the vaginal-insertion portion 50 side, and receives an ultrasonic wave returning after being reflected. Each of the ultrasonic sensors 71, 72, and 73, for example, can have an ultrasonic transducer fabricated by forming electrodes on both sides of a piezoelectric body composed of PZT (piezoelectric zirconate titanate).

The ultrasonic sensors 71, 72, and 73 can be arranged, side by side at regular intervals along the longitudinal direction of the urethral-insertion portion 40, on a surface of an intermediate portion of the urethral-insertion portion 40 on the vaginal-insertion portion 50 side. In this exemplary embodiment, the ultrasonic sensors 71, 72, 73 can be arranged so that the central ultrasonic sensor 72 is located in the same position as the puncture path of the needle tip of the puncture needle 31, in the longitudinal direction of the urethral-insertion portion 40. The ultrasonic sensors 71, 72, 73 can be so arranged that center axes (broken-line arrows in FIG. 1A) of the ultrasonic waves transmitted from the ultrasonic sensors 71, 72, 73 orthogonally intersect the axis of the urethral-insertion portion 40, which can help ensure that the ultrasonic sensors 71, 72, 73 can transmit the ultrasonic waves in a downward direction, for example, toward the puncture path on the vaginal-insertion portion 50.

In addition, the ultrasonic sensors 71, 72, and 73 can be electrically connected through the cables 6 to signal transmission-reception sections 135, 136, and 137 of the control and display unit 13, respectively.

In accordance with an exemplary embodiment, the number of the ultrasonic sensors is not limited to three, for example, the number may be one or two or may be four or more. In addition, where a plurality of ultrasonic sensors are arranged, there may be used, for example, a multi-array tip in which sensing parts of ultrasonic sensor are arrayed in columns or in rows. Besides, naturally, the layout of the ultrasonic sensors is not restricted to the above-mentioned.

In addition, the slot 411 and a slot 412 electrically connected to the cables 6 can be respectively provided at an upper end portion and a lower end portion of the supporting portion 41. The connector 221 of the movable portion 22 of the puncture needle guide member 2 can be connected to the slot 411 in a freely detachable manner. By this connection between the connector 221 and the slot 411, the urethral-insertion member 4 and the puncture needle guide member 2 can be electrically and mechanically connected to each other. In addition, a connector 531 of a movable portion 53 of the vaginal-insertion member 5 to be described later can be connected to the slot 412 in a freely detachable manner. By this connection between the connector 531 and the slot 412, the urethral-insertion member 4 and the vaginal-insertion member 5 can be electrically and mechanically connected to each other.

The couple of the slot 411 and the connector 221 and the couple of the slot 412 and the connector 531 can be different from each other in shape, such that the slot 411 and the connector 531 cannot be connected to each other, and, similarly, the slot 412 and the connector 221 cannot be connected to each other. In addition, the slot 411 and the connector 221 cannot, and the slot 412 and the connector 531 cannot, be connected to each other in the reverse sense in the longitudinal direction. Owing to these design features, misconnection between these components can be prevented.

In addition, the memory 75 can be disposed inside the supporting portion 41 as a storage section for storing various kinds of information (see FIG. 3). The memory 75 can be electrically connected to the control section 131 of the control and display unit 13 via the cable 6.

In accordance with an exemplary embodiment, for example, intrinsic information of the urethral-insertion member 4 can be stored in the memory 75. Examples of the intrinsic information on the urethral-insertion member 4 can include an index indicating whether the urethral-insertion member 4 is an unused one or a used one, the index being information indicative of whether or not the connector 221 and the slot 411 have been connected to each other at least once or information indicative of whether or not the connector 531 and the slot 412 of the urethral-insertion member 4 have been connected to each other at least once. In accordance with an exemplary embodiment, the various kinds of information stored in the memory 75 can be transmitted through the cable 6 to the control section 131 of the control and display unit when the connector 221 and the slot 411 are connected to each other or when the connector 531 and the slot 412 are connected to each other. When there is no history of connection between the connector 221 and the slot 411 and there is no history of connection between the connector 531 and the slot 412, the control section 131 determines that the urethral-insertion member 4 is an unused urethral-insertion member 4, and when there is a history of at least one-time connection in regard of either one of the two couples, the control section 131 determines that the urethral-insertion member 4 is a used urethral-insertion member 4.

The vaginal-insertion member 5 can include the vaginal-insertion portion 50, and a main body section 51 having a supporting portion 52 (which supports the vaginal-insertion portion 50) and the movable portion 53. In this exemplary embodiment, the vaginal-insertion portion 50 can be firmly attached to the supporting portion 52. In addition, the movable portion 53 can be disposed to be movable relative to the supporting portion 52, for example, vertically in FIGS. 1A and 1B. In accordance with an exemplary embodiment, a lower end portion of the movable portion 53 can be inserted inside an upper end portion of the supporting portion 52.

The vaginal-insertion portion 50 can be formed of a non-flexible rigid material, and can be straight, bar-like in shape. In addition, a distal portion of the vaginal-insertion portion 50 can be rounded, which helps permit smooth insertion of the vaginal-insertion portion 50 into the vagina.

In addition, the vaginal-insertion portion 50 can be disposed on the lower side in FIGS. 1A and 1B of the urethral-insertion portion 40 at a predetermined distance from the urethral-insertion portion 40, in such a manner that the axis of the vaginal-insertion portion is parallel to the axis of the urethral-insertion portion 40.

In accordance with an exemplary embodiment, the vaginal-insertion member 5 can have a male screw 54, and a wall portion of an upper end portion of the supporting portion 52 can be formed with a female screw 521 for engagement with the male screw 54. When the male screw 54 is rotated in a predetermined direction, the tip of the male screw 54 is pressed against the movable portion 53, thereby inhibiting the movable portion 53 from moving relative to the supporting portion 52. When the male screw 54 is rotated in a direction reverse to the above, the tip of the male screw 54 is separated from the movable portion 53, thereby permitting the movable portion 53 to move relative to the supporting portion 52. The rotation of the male screw 54 can help enable an adjustment of the separated distance between the vaginal-insertion portion 50 and the puncture needle guide portion 21, for example, the separated distance between the vaginal-insertion portion 50 and the axial portion 33 of the puncture member 3. For example, the position of the puncture needle 31 can be changed relative to the vaginal-insertion portion 50.

In accordance with an exemplary embodiment, the male screw 54 and the female screw 521 constitute a lock portion for change-over between a state in which the movable portion 53 is movable relative to the supporting portion 52 and a state in which the movable portion 53 is inhibited from moving. In addition, the supporting portion 52, the movable portion 53, the male screw 54, and the female screw 521 constitute a puncture path changing section for changing a puncture path.

In addition, as a detection section for detecting the positional relationship between the urethral-insertion portion 40 and the vaginal-insertion portion 50, a position sensor 78 for detecting the position of the movable portion 53 relative to the supporting portion 52 can be disposed inside the vaginal-insertion member 5 (see FIG. 3). The position sensor 78 can be, for example, one whose electric resistance varies according to the position of the movable portion 53 relative to the supporting portion 52. In accordance with an exemplary embodiment, the results of detection by the position sensor 78 can be transmitted to the control section 131 of the control and display unit through the cable 6 in a state where the connector 531 (described later) and the slot 412 can be in connection with each other.

In addition, a memory 76 can be disposed inside the movable portion 53 (see FIG. 3). In accordance with an exemplary embodiment, the memory 76 can be configured as a storage section for storing various kinds of information.

The connector 531 electrically connected to the position sensor 78 and the memory 76 can be provided at an upper end portion of the movable portion 53.

In accordance with an exemplary embodiment, the memory 76 can store, for example, intrinsic information of the vaginal-insertion member 5. Examples of the intrinsic information on the vaginal-insertion member 5 can include an index indicating whether the vaginal-insertion member 5 is an unused one or a used one, and which index may be information indicative of whether or not the connector 531 and the slot 412 of the urethral-insertion member 4 have been connected to each other at least once. The various kinds of information stored in the memory 76 can be transmitted to the control section 131 of the control and display unit through the cable 6 when the connector 531 and the slot 412 are connected to each other. When there is no history of connection between the connector 531 and the slot 412, the control section 131 can determine that the vaginal-insertion member 5 is an unused vaginal-insertion member 5. In accordance with an exemplary embodiment, when there is a history of at least one-time connection between the connector 531 and the slot 412, the control section 131 can determine that the vaginal-insertion member 5 is a used vaginal-insertion member 5.

The materials constituting the vaginal-insertion member 5, the urethral-insertion member 4, and the puncture needle guide member 2 are respectively not specifically restricted; for example, various resin materials can be used.

In accordance with an exemplary embodiment, the puncture member 3 has the axial portion 33, as a rotational axis of the puncture member 3, rotatably disposed in the puncture needle guide portion 21 of the puncture needle guide member 2.

In addition, the axial portion 33 can be disposed over the urethral-insertion portion 40 at a predetermined distance from the urethral-insertion portion 40 so that its axis is parallel to the axis of the urethral-insertion portion 40. In accordance with an exemplary embodiment, the axial portion 33, the urethral-insertion portion 40, and the vaginal-insertion portion 50 can be arranged on a straight line as viewed from the axial direction of the axial portion 33.

The axial portion 33 penetrates the puncture needle guide member 2 in the left-right direction in FIG. 1A. The axial portion 33 can be formed with a flange 331 and a flange 332 on the distal side and the proximal side of the puncture needle guide portion 21, with the puncture needle guide portion 21 interposed therebetween the flange 331 and the flange 332. The flanges 331 and 332 inhibit the axial portion 33 from moving in the axial direction relative to the puncture needle guide portion 21.

The puncture needle 31 can have a sharp needle tip at the distal end of the needle 31, and can be bent in an arc shape centered on the axial portion 33. In FIG. 1A, the axis of the puncture needle 31 and the axis of the axial portion 33 intersect orthogonally, which can help ensure that when the puncture member 3 is rotationally moved, the needle tip of the puncture needle 31 is moved along the arc in a plane orthogonal to the axis of the axial portion 33, namely, in a plane to which the axis is a normal.

In addition, while the needle tip of the puncture needle 31 is oriented counterclockwise in FIG. 1B in this embodiment, this is not restrictive. The needle tip may be oriented clockwise in FIG. 1B.

The puncture needle 31 may be solid or may be hollow in a tubular shape.

In addition, in this exemplary embodiment, the puncture needle 31 can be disposed on the more proximal side than a distal portion of the urethral-insertion portion 40, in the axial direction of the urethral-insertion portion 40.

In accordance with an exemplary embodiment, the puncture needle 31 may be disposed at the same position as the distal portion of the urethral-insertion portion 40 or disposed on the more distal side than the distal portion of the urethral-insertion portion 40, in the axial direction of the urethral-insertion portion 40.

In accordance with an exemplary embodiment, the puncture needle guide portion 21 can determine a puncture path for the needle tip of the puncture needle 31 by restricting the positional relationship between the puncture member 3 and the urethral-insertion portion 40 so that the needle tip of the puncture needle 31 passes a far-side from a center 311 of the puncture needle 31 compared with the urethral-insertion portion 40 or an extended line of the urethral-insertion portion 40 when the puncture member 3 moves rotationally and punctures a living body tissue. The center 311 of the puncture needle 31 can be the center of the arc shape of the puncture needle 31, for example, the rotation center of the puncture needle 31 (the puncture member 3).

Furthermore, the puncture needle guide portion 21 can determine the puncture path by restricting the positional relationship between the puncture member 3 and the vaginal-insertion portion 50 so that the needle tip of the puncture needle 31 does not collide on the vaginal-insertion member 5 or an extended line of the vaginal-insertion member 5 when the puncture member 3 moves rotationally and punctures a living body tissue.

For example, the puncture needle guide portion 21 can determine the puncture path by restricting the positional relationships between the puncture member 3 and the urethral-insertion portion 40 and the vaginal-insertion portion 50 so that the needle tip of the puncture needle 31 can pass between the urethral-insertion portion 40 or the extended line of urethral-insertion portion 40 and the vaginal-insertion portion 50 or the extended line of the vaginal-insertion portion 50 when the puncture member 3 moves rotationally and punctures a living body tissue.

In accordance with an exemplary embodiment, the puncture needle guide portion 21 can help ensure that the puncture needle 31 can puncture a living body tissue while avoiding the urethra and the vaginal wall and that the puncture needle 31 can be prevented from puncturing the urethra and from puncturing the vaginal wall.

In addition, since the puncture path can be determined, the operator can be prevented from puncturing his/her fingertip with the puncture needle 31. Thus, the safety of the operator can be relatively secured.

In accordance with an exemplary embodiment, a center angle θ1 of the arc of the puncture needle 31 is not particularly limited, but can be appropriately set according to various conditions. The center angle θ1 can be, for example, so set that when a living body tissue is punctured by the puncture needle 31, the puncture needle 31 can enter the patient's body via a body surface on one side, pass under the urethra and protrude to the exterior of the body via a body surface on the other side.

For example, the center angle θ1 of the arc of the puncture needle 31 can be, for example, about 120 to 270 degrees, for example, 160 to 230 degrees, and, for example, preferably 180 to 210 degrees.

In accordance with an exemplary embodiment, this can help ensure that when the puncture needle 31 punctures a living body tissue, the puncture needle 31 reliably enters a patient's body via a body surface on one side, passes under a urethra and protrudes to the outside of the body via a body surface on the other side.

In addition, a distal portion of the puncture needle 31 can be formed with a through-hole 312. The through-hole 312 can penetrate the puncture needle 31 in a direction perpendicular to the axis of the puncture needle 31. In accordance with an exemplary embodiment, either one of the strings 91 and 92 fixed to the implant 8 aforementioned can be inserted into the through-hole 312 and disengageably held (see FIG. 9).

A proximal portion of the axial portion 33 can be provided with a grasping unit 34 as an operating portion for a rotary movement operation of the puncture member 3. The shape of the grasping unit 34 can be a rectangular parallelepiped shape in this exemplary embodiment. At the time of putting the puncture member 3 into a rotary movement, the grasping unit 34 can be grasped with fingers and rotationally moved in a predetermined direction. The shape of the grasping unit 34 is not restricted to the disclosed embodiments.

The material constituting the puncture member 3 is not specifically restricted; for example, various metallic materials such as stainless steel, aluminum or aluminum alloys, titanium or titanium alloys, etc. can be used.

As shown in FIG. 3, the control and display unit 13 can include the control section 131, the operation section 132 for performing various operations, a display section 133 for displaying various kinds of information, and signal transmission-reception sections 135, 136, and 137 for transmission and reception of electrical signals to and from the ultrasonic sensors 71, 72, and 73. The transmission-reception sections 135, 136, and 137 of the control and display unit 13 can be electrically connected through cables to the ultrasonic sensors 71, 72, and 73, respectively.

The control section 131 can be composed, for example, of a personal computer or the like. The control section 131 receives input signals from the operation section 132, and controls the puncture apparatus 1 as a whole, inclusive of the display section 133, the signal transmission-reception sections 135 to 137, and so on. Information stored in the memories 74 to 76 can be inputted from the memories 74 to 76 to the control section 131, and the results of detection by the position sensors 77 and 78 can be inputted to the control section 131. In accordance with an exemplary embodiment, a main function of the image data production section can be accomplished by the control section 131.

The display section 133 is not specifically restricted, and examples of the display section 122 can include liquid crystal displays and CRTs (cathode ray tubes).

The signal transmission-reception sections 135 to 137 are sections which transmit to the ultrasonic sensors 71 to 73 electrical signals for actuating the ultrasonic sensors 71 to 73 to transmit ultrasonic waves and which receive electrical signals transmitted from the ultrasonic sensors 71 to 73 upon reception of ultrasonic waves (reflected ultrasonic waves) by the ultrasonic sensors 71 to 73. The electrical signals thus received are outputted to the control section 131 from the signal transmission-reception sections 135 to 137, respectively.

In accordance with an exemplary embodiment, the control section 131 controls the signal transmission-reception sections 135 to 137, performs transmission and reception of ultrasonic waves by the ultrasonic sensors 71 to 73, and produces image data for displaying on the display section 133 an ultrasonic image based on the signals transmitted from the signal transmission-reception sections 135 to 137, for example, based on the information acquired by the ultrasonic sensors 71 to 73. Then, the control section 131 can display the ultrasonic image on the display section 133.

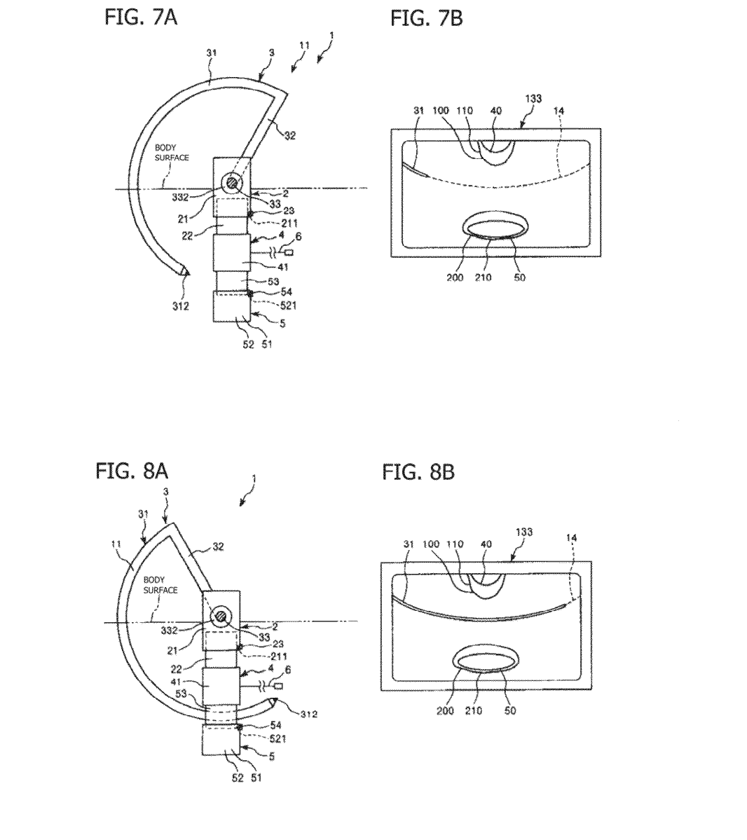

The ultrasonic image can be produced by a method in which distances from the ultrasonic sensors 71 to 73 to an ultrasonic wave-reflecting object are measured based on the periods of time after the transmission of ultrasonic waves from the ultrasonic sensors 71 to 73 until returning of reflected waves to the ultrasonic sensors 71 to 73, and the object is visualized based on the measured distances while taking into account the intensities of the reflected waves, etc. The ultrasonic image can include the patient's vagina and urethra, the vaginal-insertion portion 50 of the puncture apparatus 1, the puncture needle 31 of the puncture member 3, and the like (see FIG. 7B). The image data concerning the ultrasonic image can be data for displaying the ultrasonic image on the display section 133.

In addition, the control section 131 can determine a predicted path obtained by predicting a puncture path of the needle tip of the puncture needle 31 at the time of puncturing a living body tissue with the puncture member 3, and can produce image data for displaying on the display section 133 an image of the predicted path. Then, the control section 131 can display the image of the predicted path on the display section 133 in the state of being superimposed on the above-mentioned ultrasonic image.

The predicted path of the puncture path can be obtained based on the radius r of the arc of the puncture needle 31 (see FIG. 1B) and the positional relationship between the urethral-insertion portion 40 (the ultrasonic sensors 71 to 73) and the puncture needle 31, for example, the separated distance between the urethral-insertion portion 40 and the puncture needle guide portion 21.

The term “image data” used hereinafter includes the image data concerning the predicted path of the puncture path and the image data concerning the ultrasonic image.

In addition, in this exemplary embodiment, the image to be displayed on the display section 133 can be formed by taking a central portion of an upper portion of the displayed image on the display section 133 as the position of the urethral-insertion portion 40, for example, the positions of the ultrasonic sensors 71 to 73. In accordance with an exemplary embodiment, an image of the urethral-insertion portion 40 cannot be obtained from the information acquired by the ultrasonic sensors 71 to 73. However, the shape, size, and position of the urethral-insertion portion 40 are preliminarily known. Based on the preliminarily known factors, the image of the urethral-insertion portion 40 can be predicted and can be displayed on the display section 133 (see FIG. 4C). In accordance with an exemplary embodiment, the image thus predicted of the urethral-insertion portion 40 may naturally be omitted.

Now, an exemplary method of using the puncture apparatus 1 will be described below referring to FIGS. 4A to 9. Here, a procedure taken until an implant 8 for treatment of female urinary incontinence is implanted in a living body by use of the puncture apparatus 1 will be described.

First, as shown in FIGS. 1A, 1B, and 2, the connector 221 is inserted into the slot 411, and the puncture needle guide member 2 is mounted to the urethral-insertion member 4. In addition, the connector 531 is inserted into the slot 412, and the vaginal-insertion member 5 is mounted to the urethral-insertion member 4. As a result, the puncture apparatus 1 is in an assembled state.

The various kinds of information stored in the memories 74 to 76 and the results of detection by the position sensors 77 and 78 are transmitted to the control section 131 of the control and display unit. Based on the information, the control section 131 determines whether the puncture member 3, the urethral-insertion member 4, and the vaginal-insertion member 5 are unused ones or used ones, respectively. Where any of the members is a used one, an alarm can be displayed on the display section 133, which can help avoid erroneous use of a used member. In addition, based on the results of detection by the position sensor 77 and the radius r of the arc of the puncture needle 31, the control section 131 can obtain a predicted path 14 of the puncture path for the needle tip of the puncture needle 31.

Next, as shown in FIGS. 4A and 4B, the puncture apparatus main body 11 of the puncture apparatus 1 is mounted onto a patient. For example, the urethral-insertion portion 40 of the puncture apparatus main body 11 is inserted into a urethra 100 of the patient, and the vaginal-insertion portion 50 is inserted into a vagina 200 of the patient. In this instance, the mounting is so conducted that the marker 42 is located in a urethral orifice or in front of the urethral orifice, such that a distal portion of the urethral-insertion member 4 can be disposed in front of a bladder. A gap between a urethral wall and the urethral-insertion portion 40 and a gap between a vaginal wall and the vaginal-insertion portion 50 can each be filled, for example, with physiological saline solution, a gel for ultrasonic observation, or the like.

As shown in FIG. 4C, the vagina 200 and a vaginal wall 210, the urethra 100 and a urethral wall 110, the vaginal-insertion portion 50, the urethral-insertion portion 40, the predicted path 14 of the puncture path for the needle tip of the puncture needle 31, and so on are displayed on the display section 133. In accordance with an exemplary embodiment, looking at an image displayed on the display section 133, the operator can grasp whether the puncture path for the needle tip of the puncture needle 31 is appropriate or inappropriate. The predicted path 14 is indicated, for example, with broken line as illustrated, or in a color different from the color of other parts, or the like manner, for clear visibility.

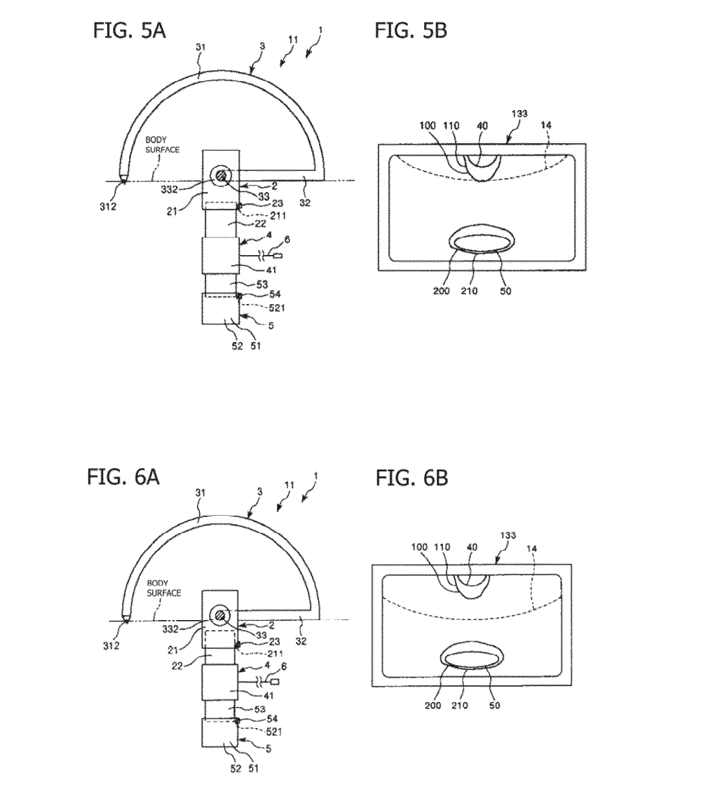

Subsequently, as shown in FIGS. 5A and 5B, the separated distance between the urethral-insertion portion 40 and the vaginal-insertion portion 50 is enlarged, to enlarge the separated distance between the urethra 100 and the vagina 200. In accordance with an exemplary embodiment, this operation may be omitted. The separated distance between the urethral-insertion portion 40 and the vaginal-insertion portion 50 is enlarged by moving the puncture needle guide portion 21 upward relative to the movable portion 22, or by moving the supporting portion 52 downward relative to the movable portion 53, or by both of these movements. FIG. 5A shows a case where the supporting portion 52 is moved downward relative to the movable portion 53.

Here, where the predicted path 14 is appropriate, namely, where the predicted path 14 is located between the urethra 100 and the vagina 200 and is sufficiently spaced apart from the urethral wall 110 and the vaginal wall 210 as shown in FIG. 6B, a puncturing operation by the puncture member 3 is carried out.

However, where the predicted path 14 is inappropriate, for example, where the predicted path 14 intersects the urethral wall 110 as shown in FIG. 5B, where the predicted path 14 intersects the vaginal wall 210, where the predicted path 14 is too close to the urethral wall 110, or where the predicted path 14 is too close to the vaginal wall 210, the predicted path can be changed so as to be appropriate. In accordance with an exemplary embodiment, a configuration may be adopted in which, for example, alarm information is displayed on the display section 133 when the predicted path 14 is inappropriate.

Where the predicted path 14 intersects the urethral wall 110 as shown in FIG. 5B or where the predicted path 14 is too close to the urethral wall 110, the puncture needle guide portion 21 is moved downward relative to the movable portion 22 as shown in FIG. 6A. This results in that the predicted path 14 is sufficiently spaced apart from the urethral wall 110 and the vaginal wall 210, and is therefore appropriate, as shown in FIG. 6B.

Also, though not illustrated, where the predicted path 14 intersects the vaginal wall 210 or where the predicted path 14 is too close to the vaginal wall 210, the puncture needle guide portion 21 can be moved upward relative to the movable portion 22, or the supporting portion 52 can be moved downward relative to the movable portion 53, or both of these movements can be carried out.

In accordance with an exemplary embodiment, the puncture member 3 may be replaced when the predicted path 14 is appropriate. The replacement of the puncture member 3 can be accompanied by replacement of the puncture needle guide member 2.

As shown in FIG. 7A, with the grasping unit 34 grasped, the puncture member 3 is rotated counterclockwise in FIG. 7A, which can result in that the needle tip of the puncture needle 31 is moved counterclockwise in FIG. 7A along the arc of the needle tip of the puncture needle 31, and the puncture needle 31 is displayed on the display section 133 as shown in FIG. 7B. Accordingly, the operator can recognize that the puncture path for the needle tip of the puncture needle 31 is appropriate.

Then, as shown in FIGS. 8A and 9, the puncture member 3 is rotated further. Upon this rotation, the needle tip of the puncture needle 31 can puncture a body surface of an inguinal region on the left side in FIG. 8A of the patient or of a region in the vicinity of the inguinal region, to enter the body, passes an obturator foramen on the left side in a pelvis, can pass under the urethra 100, for example, between the urethra 100 and the vagina 200, can pass an obturator foramen on the right side in the pelvis, and can protrude to the outside of the body via a body surface of an inguinal region on the right side or of a region in the vicinity of the inguinal region. As a result, a puncture hole in which to implant the implant 8 in the living body can be formed in the patient. This puncture hole can be a through-hole extending from the body surface of the inguinal region on the left side in FIG. 8A or of the region in the vicinity of the inguinal region, passes the obturator foramen on the left side in the figure, passes between the urethra 100 and the vagina 200, and passes the obturator foramen on the right side in the figure, to reach the body surface of the inguinal region on the right side in the figure or of the region in the vicinity of the inguinal region.

Subsequently, as shown in FIG. 9, an end portion of either one of the strings 91 and 92 can be fixed to the implant 8, in the illustrated configuration, the string 91, is inserted into and passed through the through-hole 312 in the puncture needle 31. Consequently, the end portion of the string 91 is held on a distal portion of the puncture needle 31.

Next, with the grasping unit 34 grasped, the puncture member 3 is rotated clockwise in FIG. 9. As a result, the needle tip of the puncture needle 31 is moved clockwise in FIG. 9 along the arc of the needle tip of the puncture needle 31, enters the patient's body via the body surface of the inguinal region on the right side in FIG. 9 or the region in the vicinity of the inguinal region, passes the obturator foramen on the right side in the figure of the pelvis, passes under the urethra 100, specifically between the urethra 100 and the vagina 200, and passes the obturator foramen on the left side in the figure of the pelvis, to come out of the body via the body surface in the inguinal region on the left side or the region in the vicinity of the inguinal region. For example, the puncture needle 31 can be pulled out of the body.

Subsequently, the end portion of the string 91 is pulled out of the through-hole 312 of the puncture needle 31. In addition, the puncture apparatus main body 11 is detached from the patient. Specifically, the urethral-insertion portion 40 is pulled out of the urethra 100, and the vaginal-insertion portion 50 is pulled out of the vagina 200 of the patient.

Next, while pulling the string 92, the string 91 is pulled, the implant 8 is inserted into the through-hole formed in the patient, and a left-side end portion of the implant 8 is drawn out of the through-hole to the exterior of the body, leaving a right-side end portion of the implant 8 in the exterior of the body.

Subsequently, the strings 91 and 92 are pulled with predetermined forces respectively, whereby the position of the implant 8 relative to the urethra 100 is adjusted, unnecessary portions of the implant 8 can be cut away, and a predetermined treatment can be conducted, to complete the procedure.

As described above, according to the puncture apparatus 1, implanting of the implant 8 into a living body can be realized by only a low invasive procedure such as puncturing with the puncture needle 31, without needing invasive incision or the like. Therefore, the burden on the patient is relatively light, and the safety of the patient is relatively high.

In addition, since the urethra, the vagina, the predicted path of the puncture path for the needle tip of the puncture needle 31, and the puncture needle 31 are displayed on the display section 133, there is obtained the following advantage. By changing the puncture path so as to attain an appropriate predicted path in the case where the predicted path of the puncture path is inappropriate, a living body with the puncture needle 31 can be punctured while avoiding the urethral wall and the vaginal wall, and thereby can help prevent the puncture needle 31 from puncturing the urethral wall and from puncturing the vaginal wall. Thus, relative safety can be secured. In addition, a desired part of a living body tissue can be reliably punctured, and the implant 8 can be relatively assuredly implanted into a desired region.

In addition, since it is unnecessary for the operator himself/herself to perform incision or the like, damaging of the patient's fingertip with a surgical knife or the like can be obviated. Thus, the safety of the operator is relatively secured.

While the ultrasonic sensors are provided in the urethral-insertion portion in this embodiment, this configuration is not restrictive. The ultrasonic sensors may be provided in the vaginal-insertion portion, for example.

In addition, while the puncture hole formed in the patient by the puncture needle is a through-hole in this exemplary embodiment, this is not restrictive. For example, the puncture hole may not necessarily be of a penetrating (passing-through) type.

While the puncture needle as a whole is bent in an arc shape in this exemplary embodiment, this configuration is not restrictive. For example, the puncture needle may only be partly bent in an arc shape. Thus, for example, the puncture needle can have an arc-shaped bent region at least at a part of the puncture needle.

Further, at least a part of the puncture needle can have a bent region. For example, the puncture needle may be bent in an elliptical arc shape as a whole, or only a part of the puncture needle may have an elliptical arc-shaped portion. Thus, in accordance with an exemplary embodiment, at least a part of the puncture needle assembly may have an elliptical arc-shaped bent region.

In accordance with an exemplary embodiment, the puncture needle may lack a bent region. For example, the puncture needle may be straight.

In addition to the display section, the puncture apparatus 1 may have, for example, a buzzer or the like as an alarm section which operates when the predicted path of the puncture path is inappropriate.

FIG. 10 is a side view showing a second exemplary embodiment of the puncture apparatus according to the present disclosure. The left side in FIG. 10 will be hereinafter referred to as the “distal end,” the right side in the figure as the “proximal end,” the upper side in the figure as the “upper side,” and the lower side in the figure as the “lower side.”

The second exemplary embodiment will be described below mainly regarding its differences from the aforementioned first embodiment, and descriptions of the same or similar items to those described above will be omitted.

As shown in FIG. 10, in a puncture apparatus 1 in the second exemplary embodiment, an axis 333 of an axial portion 33 of a puncture member 3 is inclined against an axis 43 of a urethral-insertion portion 40 so that the separated distance between the axis 333 and the axis 43 increases distally, which can help enable an implant 8 to be implanted in an inclined state. In addition, depending on the patient, a body surface may be somewhat inclined against the urethra, and the body surface and obturator foramens may be substantially parallel. In this case, the inclination of the axis 333 can enable the axial portion 33 of the puncture member 3 to be parallel to and close to the body surface and the obturator foramens, which can help ensure that the distance between the puncture needle 31 and the obturator foramen can be reduced, and positioning of the puncture apparatus 1 for passing the puncture needle 31 through the obturator foramens can be carried out relatively easily and accurately.

In accordance with an exemplary embodiment, the axis 43 of the urethral-insertion portion 40 and the axis of a vaginal-insertion portion 50 are parallel, and the axis 333 of the axial portion 33 of the puncture member 3 can be inclined against the axis of the vaginal-insertion member 5 so that the distance between the axis 333 and the axis of the vaginal-insertion portion 50 increases distally.

An inclination angle θ2 of the axis 333 against the axis 43 is not particularly limited, and can be, for example, about 3 to 60 degrees, and for example, preferably 30 to 45 degrees, which can help enable securer support of the urethra by the implant 8.

In addition, ultrasonic sensors 71, 72, 73 can be arranged that the ultrasonic sensor 72 in the middle is located on a puncture path of a needle tip of the puncture needle 31 in side view shown in FIG. 10. The ultrasonic sensors 71, 72, 73 can be arranged that center axes (broken-line arrows in FIG. 10) of ultrasonic waves transmitted from the ultrasonic sensors 71, 72, 73 can be inclined against the axis 43 of the urethral-insertion portion 40, which can help ensure that the ultrasonic sensors 71, 72, 73 transmit the ultrasonic waves toward a leftward and downward side in FIG. 10, for example, toward the puncture path on the vaginal-insertion portion 50

According to this puncture apparatus 1, similar effects to those obtained in the first exemplary embodiment above can be obtained.

The axis 333 of the axial portion 33 of the puncture member 3 may be inclined against the axis 43 of the urethral-insertion portion 40 so that the separated distance between the axis 333 and the axis 43 decreases distally.

In addition, the axis 43 of the urethral-insertion portion 40 and the axis of the vaginal-insertion portion 50 may not necessarily be parallel.

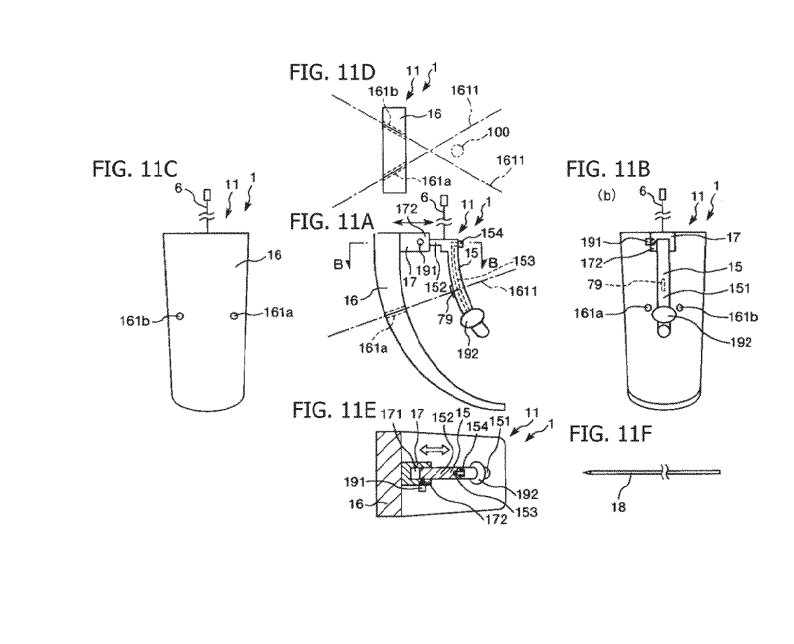

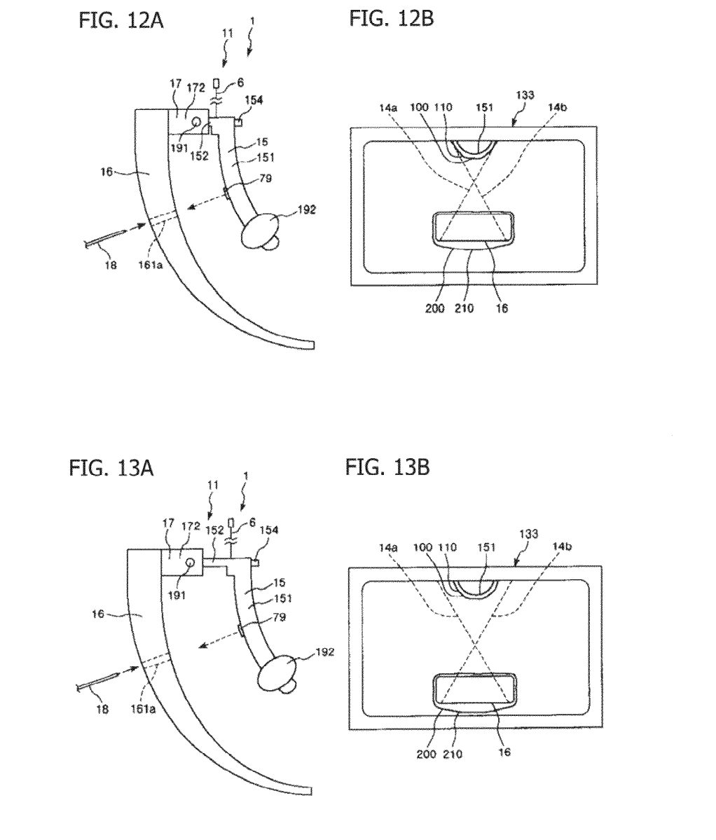

FIGS. 11A to 11F are views illustrating a third exemplary embodiment of the puncture apparatus according to the present disclosure, wherein FIG. 11A is a side view, FIG. 11B is a front view, FIG. 11C is a back elevation, FIG. 11D is a plan view showing a vaginal-insertion portion, FIG. 11E is a sectional view taken along line B-B in FIG. 11A, and FIG. 11F is a side view showing a puncture needle. FIGS. 12A to 13B are views for explaining an operating procedure of the puncture apparatus shown in FIGS. 11A to 11F, wherein FIGS. 12A and 13A are side views, and FIGS. 12B and 13B are views showing an image displayed on a display section. In FIGS. 12A and 13A, the same symbols as those for actual (or real) parts are used in the image displayed on the display section.

In the following description, the lower side and the upper side along the longitudinal direction of a vaginal-insertion portion and a urethral-insertion portion in FIGS. 11A, 12A, and 13A will be referred to as the “distal end” and the “proximal end,” respectively. Besides, in FIG. 11D, the urethra of a patient in the case where a puncture apparatus main body of the puncture apparatus is assumed to be mounted onto the patient is drawn in an alternate long and two dot dashes line.

The third exemplary embodiment will be described below mainly regarding its differences from the aforementioned first embodiment, and descriptions of the same or similar items to those described above will be omitted.

As shown in FIGS. 11A to 11F, in a puncture apparatus 1 according to the third exemplary embodiment, a puncture apparatus main body 11 can include a urethral-insertion member 15 having an elongated urethral-insertion portion 151 which is inserted into a urethra, an elongated vaginal-insertion portion 16 which is inserted into a vagina, a interlock portion 17 as an interlocking section for interlocking the vaginal-insertion portion 16 and the urethral-insertion member 15, and a puncture needle 18. The puncture needle 18 is designed to penetrate a vaginal wall from the vagina and puncture a living body tissue in the vicinity of the urethra and the vagina. While the puncture needle 18 is straight in shape, it may be curved.

The shape of the vaginal-insertion portion 16 is not specifically restricted so long as it is elongated. For example, the shape of the vaginal-insertion portion 16 can be a plate-like shape. In accordance with an exemplary embodiment, the width of the vaginal-insertion portion 16 gradually decreases distally, and the thickness of the vaginal-insertion portion 16 gradually decreases distally. In accordance with an exemplary embodiment, a distal portion of the vaginal-insertion portion 16 can be rounded, which can help enhance safety of the patient.

In accordance with an exemplary embodiment, the vaginal-insertion portion 16 can be curved, which can help ensure that when the vaginal-insertion portion 16 is inserted in the vagina, the operator can orient a field of operation to the front side, thereby enlarging the field of operation.

In accordance with an exemplary embodiment, an intermediate part in the longitudinal direction of the vaginal-insertion portion 16, or a central part of the vaginal-insertion portion 16, can constitute a puncture needle guide portion. The central part of the vaginal-insertion portion 16 can be formed with two guide holes 161a and 161b, which penetrate the vaginal-insertion portion 16 in the thickness direction of the two guide holes 161a and 161b. The puncture needle 18 can be used by being inserted in each of the guide holes 161a and 161b.

Each of the guide holes 161a and 161b can be configured to support the puncture needle 18 in a longitudinally movable manner so as to guide the puncture needle 18 in the vicinity of the urethral-insertion portion 151 and in a direction for avoiding the urethral-insertion portion 151, for example, in the vicinity of the urethra and in a direction for avoiding the urethra. In accordance with an exemplary embodiment, the guide holes 161a and 161b can be configured to guide two puncture needles 18 in different directions, respectively.

In addition, while the shapes of the guide holes 161a and 161b are straight in shape respectively in this embodiment, this is not specifically restricted. For example, where the puncture needles 18 are bent, the guide holes 161a and 161b are each preferably bent.

In accordance with an exemplary embodiment, each of extended lines 1611 of the guide holes 161a and 161b does not intersect the urethral-insertion portion 151. For example, the extended lines 1611 can be deviated from the urethral-insertion portion 151, which can help ensure that when each of the puncture needles 18 inserted in the guide holes 161a and 161b is moved in the distal direction of the guide holes 161a and 161b, the puncture needle 18 would not contact the urethral-insertion portion 151, for example, would not contact the urethra. Thus, the puncture needle 18 can pass the vicinity of the urethral-insertion portion 151 while avoiding the urethral-insertion portion 151, for example, the puncture needle 18, can pass the vicinity of the urethra while avoiding the urethra. Consequently, the puncture needle 18 can avoid damaging the urethra.

The extended lines 1611 of the guide holes 161a and 161b intersect each other between the urethral-insertion portion 16 and the vaginal-insertion portion 151.

The urethral-insertion member 15 has the urethral-insertion portion 151, and a protruding portion 152 formed at a proximal portion of the urethral-insertion portion 151 to project from the urethral-insertion portion toward the left side in FIG. 11A.

The shape of the urethral-insertion portion 151 is not specifically restricted so long as it is elongated, for example, in this exemplary embodiment, the shape is a bar-like shape. In accordance with an exemplary embodiment, a distal portion of the urethral-insertion portion 151 can be rounded, which helps enhance safety of the patient.

In addition, the urethral-insertion portion 151 can be curved in the same direction as the vaginal-insertion portion 16. The curvature of the urethral-insertion portion 151 can be set equal to that of the vaginal-insertion portion 16. In accordance with an exemplary embodiment, the posture of the urethral-insertion portion 151 is so set that the separated distance between the urethral-insertion portion 151 and the vaginal-insertion portion 16 is constant along the longitudinal direction of the urethral-insertion portion 151.

The interlock portion 17 can be fixed to the right side, in FIG. 11A, of a proximal portion of the vaginal-insertion portion 16.

The interlock portion 17 is formed with a bottomed hole 171 along the left-right direction in FIG. 11A. The hole 171 is open on the right side in FIG. 11A.

The protruding portion 152 of the urethral-insertion member 15 is inserted in the hole 171 in a longitudinally movable manner. With the urethral-insertion member 15 moved in the longitudinal direction of the protruding portion 152 relative to the interlock portion 17, the separated distance between the vaginal-insertion portion 16 and the urethral-insertion portion 151 is changed. Therefore, the hole 171 in the interlock portion 17 and the protruding portion 152 of the urethral-insertion member 15 constitute an adjustment section for adjusting the separated distance between the vaginal-insertion portion 16 and the urethral-insertion portion 151, and also constitute a puncture path changing section. Since patients have individual differences and the separated distance between the vagina and the urethral may vary from patient to patient, the separated distance between the vaginal-insertion portion 16 and the urethral-insertion portion 151 can be adjusted, as required, to be suitable for the patient to be treated.

In accordance with an exemplary embodiment, irrespectively of how the separated distance between the vaginal-insertion portion 16 and the urethral-insertion portion 151 is set, it can be ensured that the extended lines 1611 of the guide holes 161a and 161b are deviated from the urethral-insertion portion 151 and that the extended lines intersect each other between the vaginal-insertion portion 16 and the urethral-insertion portion 151 even when the vaginal-insertion portion 16 and the urethral-insertion portion 151 are set most closely.

In addition, the puncture apparatus main body 11 can have a male screw 191. The interlock portion 17 is formed, in its part corresponding to the hole 171 in the end portion on the right side in FIG. 11A, with a female screw portion 172 having a female screw for engagement with the male screw 191.

When the male screw 191 is rotated in a predetermined direction, a tip of the male screw 191 is pressed against the protruding portion 152 of the urethral-insertion member 15, whereby the urethral-insertion member 15 is inhibited from moving relative to the interlock portion 17. When the male screw 191 is rotated in a direction reverse to the above-mentioned, the tip of the male screw 191 is separated from the protruding portion 152, thereby permitting the urethral-insertion member 15 to move relative to the interlock portion 17. The rotation of the male screw 191 can change the separated distance between the vaginal-insertion portion 16 and the urethral-insertion portion 151, and can change the puncture path for the needle tip of the puncture needle 18.

The male screw 191 and the female screw portion 172 constitute a lock portion for change-over between a state in which the urethral-insertion portion 15 is movable relative to the interlock portion 17 and a state in which such movement of the urethral-insertion member 15 is inhibited.

In accordance with an exemplary embodiment, as ultrasonic transmission-reception sections for performing transmission and reception of ultrasonic waves, a multi-array chip 79 wherein sensing sections of ultrasonic sensors are arrayed in a column pattern can be disposed on a surface on the vaginal-insertion portion 16 side of an intermediate portion of the urethral-insertion portion. The multi-array chip 79 transmits ultrasonic waves toward the vaginal-insertion portion 16 side, for example, toward the vicinity of the guide holes 161a and 161b in the vaginal-insertion portion 16.

In addition, as a restriction portion for restricting the position of the urethral-insertion portion 151 in the longitudinal direction within the urethra, an inflatable and deflatable balloon 192 can be provided at a distal portion of the urethral-insertion portion 151. At the time of using the puncture apparatus 1, the balloon 192 is inserted in a patient's bladder, and the balloon 192 is caught on a bladder neck in its inflated state, whereby the position of the urethral-insertion portion 151 relative to the bladder and the urethra can be fixed.

In accordance with an exemplary embodiment, the balloon 192 can be disposed on the more distal side than the positions of the extended lines 1611 of the guide holes 161a and 161b in the longitudinal direction of the urethral-insertion portion 151 within the urethral-insertion portion 151. By this configuration, the puncture needle 18 can be prevented from puncturing the bladder.

In addition, the urethral-insertion portion 151 can include a lumen 153. A distal end of the lumen 153 is open into the balloon 192, while a proximal end is open in a side surface of a proximal portion of the urethral-insertion portion 151. The proximal portion of the urethral-insertion portion 151 is formed with a port 154 communicating with the proximal end of the lumen 153.

A balloon-inflating instrument such as, for example, a syringe (not shown) can be connected to the port 154, a working fluid supplied from the balloon-inflating instrument is fed into the balloon 192 or drawn out of the balloon 192 through the lumen 153, whereby the balloon 192 is inflated or deflated. In accordance with an exemplary embodiment, a liquid, such as physiological saline solution, or a gas or the like can be used as the working fluid for balloon inflation.

The restriction portion for restricting the position of the urethral-insertion portion 151 within the urethra is not restricted to the balloon 192, and examples of the restriction portion other than the balloon 192 can include one in which a part of the urethral-insertion portion 151 is curved, and one which grasps a part of a surface of a urinary tract tissue.

In addition, as a detection section for detecting the positional relationship between the urethral-insertion portion 151 and the puncture needle 18, a position sensor (not shown) for detecting the position of the protruding portion 152 of the urethral-insertion member relative to the interlock portion 17 can be disposed at the interlock portion 17. In accordance with an exemplary embodiment, the position sensor can be, for example, one whose electric resistance varies according to the position of the protruding portion 152 relative to the interlock portion 17. In accordance with an exemplary embodiment, the results of detection by the position sensor can be transmitted through a cable 6 to a control section 131 of a control and display unit. Based on the detection results, the control section 131 can determine the separated distance between the urethral-insertion portion 40 and the puncture needle guide portion. Then, the control section 131 can obtain a predicted path of a puncture path, based on the positional relationship between the urethral-insertion portion 151 (the multi-array chip 79) and the puncture needle 18, for example, the separated distance between the urethral-insertion portion 151 and the puncture needle guide portion.

An exemplary method of using the puncture apparatus 1 will be described below, referring to FIGS. 12A, 12B, 13A, and 13B. Here, a description will be made of a procedure taken until an implant (not shown) for treatment of female urinary incontinence is implanted into a living body by use of the puncture apparatus 1.

First, as shown in FIG. 12A, the puncture apparatus main body 11 of the puncture apparatus 1 is mounted onto a patient. Specifically, the vaginal-insertion portion 16 of the puncture apparatus main body 11 is inserted into a vagina, and the urethral-insertion portion 151 is inserted into a urethra. As a result, the position of the urethra relative to the vaginal-insertion portion 16 is fixed to a specified position.

As shown in FIG. 12B, a vagina 200 and a vaginal wall 210, a urethra 100 and a urethral wall 110, the vaginal-insertion portion 16, the urethral-insertion portion 151, predicted paths 14a and 14b of puncture paths for the needle tips of the puncture needles 18, and the like are displayed on the display section 133. Looking at an image thus displayed on the display section 133, the operator can grasp whether the puncture paths for the needle tips of the puncture needles 18 are appropriate or inappropriate.

Here, in a case where the predicted paths 14a and 14b are appropriate, that is, where the predicted paths 14a and 14b are sufficiently separated from the urethral wall 110, as shown in FIG. 13B, a puncturing operation by the puncture needles 18 is conducted.

However, in a case where the predicted paths 14a and 14b are inappropriate, for example, where the predicted paths 14a and 14b intersect the urethral wall 110 as shown in FIG. 12B or where the predicted paths 14a and 14b are too close to the urethral wall 110, the puncture paths are changed so as to be appropriate.

Specifically, as shown in FIG. 13A, the protruding portion 152 is moved toward the left side in FIG. 13A relative to the interlock portion 17, thereby reducing the separated distances between the urethral-insertion portion 151 and the guide holes 161a and 161b, which results in that the predicted paths 14a and 14b are sufficiently separated from the urethral wall 110, and are appropriate, as shown in FIG. 13B.

Next, the puncture needle 18 is inserted into the guide hole 161a, and is moved in the distal direction of the guide hole 161a. Also, the puncture needle 18 is inserted into the guide hole 161b, and is moved in the distal direction of the guide hole 161b. As a result, two puncture holes for implanting implants 8 in the living body are formed in the patient. One of the two puncture holes is a bottomed hole or through-hole which extends from the vaginal wall, passes a region in the vicinity of the urethra and on the left side of the urethra, and extends toward an obturator foramen on the left side. The other of the two puncture holes is a bottomed hole or through-hole which extends from inside the vagina, passes a region in the vicinity of the urethra and on the right side of the urethra, and extends toward an obturator foramen on the right side. In accordance with an exemplary embodiment, the puncture holes intersect each other.

From then on, by using predetermined medical instruments such as, for example, a needle and a catheter, the implants 8 are inserted into the puncture holes, to implant the implants 8 in the living body, thereby completing the procedure.

According to this puncture apparatus 1, similar effects as those obtained in the aforementioned first embodiment can be obtained.

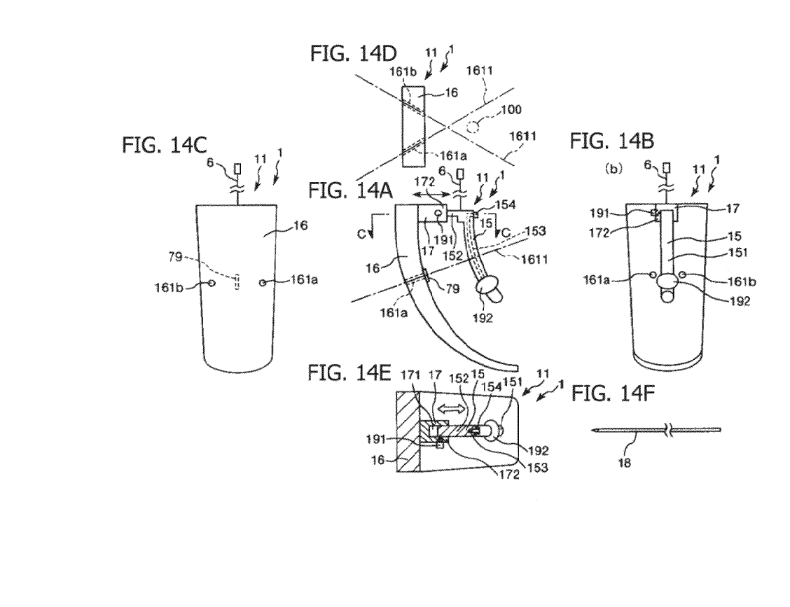

FIGS. 14A to 14F are views showing a fourth exemplary embodiment of the puncture apparatus according to the present disclosure, in which FIG. 14A is a side view, FIG. 14B is a front view, FIG. 14C is a back elevation, FIG. 14D is a plan view showing a vaginal-insertion portion, FIG. 14E is a sectional view taken along line C-C in FIG. 14A, and FIG. 14F is a side view showing a puncture needle.

In the following description, the lower side and the upper side along the longitudinal direction of a vaginal-insertion portion and a urethral-insertion portion in FIG. 14A will be referred to as the “distal end” and the “proximal end,” respectively. In addition, in FIG. 14D, the urethra of a patient in the case where a puncture apparatus main body of the puncture apparatus is mounted onto the patient is drawn in an alternate long and two short dashes line.

Now, the fourth exemplary embodiment will be described below mainly regarding its differences from the aforementioned third exemplary embodiment, and descriptions of the same or similar items to those described above will be omitted.

As shown in FIGS. 14A to 14F, in a puncture apparatus 1 according to the fourth exemplary embodiment, a multi-array chip 79 can be disposed on a surface on a urethral-insertion portion 151 side of an intermediate portion of a vaginal-insertion portion 16. The multi-array chip 79 transmits ultrasonic waves toward the urethral-insertion portion 151 side, for example, in a direction of extended lines 1611 of guide holes 161a and 161b, in side view in FIG. 14A.

In the other points than the disclosed above, the fourth exemplary embodiment is the same as the third exemplary embodiment, so the description of the same points is omitted here.

According to this puncture apparatus 1, similar effects to those obtained in the aforementioned third embodiment can be obtained.

While some illustrated embodiments of the puncture apparatus according to the present disclosure have been described above, the disclosure is not limited to the embodiments. Each of the components of the puncture apparatus may be replaced with one of an arbitrary configuration that is able to function in an equivalent manner. Besides, arbitrary configurations may be added to the aforementioned.

In addition, the puncture apparatus of the present disclosure may be a combination of arbitrary two or more configurations (features) of the above embodiments.

While the number of insertion portions is two in the above embodiments, this is not restrictive of the present disclosure. For example, only one insertion portion may be provided.

In the embodiments above, description has been made of the case where the puncture apparatus of the present disclosure is applied to an apparatus used in implanting an implant into a living body for treatment of female urinary incontinence. The application of the puncture apparatus of the present disclosure, however, is not limited to the aforementioned. The puncture apparatus of the present disclosure is also applicable, for example, to an apparatus used in implanting an implant into a living body for treatment of male urinary incontinence. Where the puncture apparatus of the present disclosure is applied to an apparatus used in implanting an implant into a living body for treatment of male urinary incontinence, the vaginal-insertion portion to be inserted into a vagina, as an insertion portion to be inserted into a living body lumen, may be replaced, for example, by a rectal insertion portion which is inserted into a rectum via an anus.

According to the present disclosure, at the time of puncturing a living body tissue with a puncture needle, puncturing of a part which should not be punctured can be prevented, and the burden on the patient is light. In addition, the safety of the patient and the safety of the operator are both high.

For example, in the case of using the puncture apparatus of the present disclosure for treatment of female urinary incontinence, the insertion portion of the puncture apparatus is inserted into the urethra, and the puncture needle is rotationally moved to puncture the living body with the puncture needle. In this instance, an image including the urethra and a predicted path of the puncture path can be displayed, which can help ensure that when the puncture needle is predicted to puncture the urethra, the puncture path can be changed to prevent the puncture needle from puncturing the urethra.

In addition, at the time of implanting an implant for treatment of urinary incontinence, the implanting operation can be carried out with a low invasive procedure, without needing incision of a vaginal wall. In addition, the operator can be prevented from damaging his/her fingertip. Accordingly, the puncture apparatus of the present disclosure has industrial applicability.

It should be understood by those skilled in the art that various modifications, combinations, sub-combinations and alterations may occur depending on design requirements and other factors insofar as they are within the scope of the appended claims or the equivalents thereof.

The detailed description above describes a puncture apparatus. The disclosure is not limited, however, to the precise embodiments and variations described. Various changes, modifications and equivalents can effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. A puncture apparatus comprising:
an elongated insertion portion configured to be inserted into a living body lumen;
a puncture needle configured to puncture a living body tissue near the insertion portion from a part different from the living body lumen, with the insertion portion inserted in the living body lumen;
a puncture needle guide portion which movably supports the puncture needle and which determines a puncture path for a needle tip of the puncture needle when the puncture needle punctures the living body tissue;
an ultrasonic transmission-reception section provided in the insertion portion or the puncture needle guide portion, for transmission and reception of an ultrasonic wave; and
an image data production section for producing image data for displaying on a display section an image including the living body lumen and a predicted path of the puncture path, based on information acquired by the ultrasonic transmission-reception section.

2. The puncture apparatus according to claim 1, comprising:
a puncture path changing section for changing the puncture path.

3. The puncture apparatus according to claim 2, wherein the puncture path changing section changes a position of the puncture needle relative to the insertion portion.

4. The puncture apparatus according to claim 3, comprising:
a detection section for detecting a positional relationship between the insertion portion and the puncture needle; and
wherein the image data production section is configured to determine the predicated path of the puncture path, based on results of detection by the detection section.

5. The puncture apparatus according to claim 2, wherein the predicted path of the puncture path is located between an urethra wall and a vaginal wall, and if the puncture path is not between the urethra wall and the vaginal wall, the puncture path is changed by the puncture path changing section so that the predicated path will be between the urethra wall and the vaginal wall.

6. The puncture apparatus according to claim 1,
wherein the puncture needle has a bent region, and is rotatably disposed in the puncture needle guide portion; and
the puncture needle guide portion is configured to determine the puncture path such that a needle tip of the puncture needle passes a far-side or a near-side from a rotation center of the puncture needle compared with the insertion portion when the puncture needle moves rotationally and punctures the living body tissue.

7. The puncture apparatus according to claim 1, wherein the puncture needle guide portion is configured to determine the puncture path such that the needle tip of the puncture needle passes a vicinity of the insertion portion while avoiding the insertion portion.

8. The puncture apparatus according to claim 1, wherein the living body lumen is a urethra or a vagina.

9. A puncture apparatus comprising:
an elongated first insertion portion configured to be inserted into a first living body lumen;
an elongated second insertion portion configured to be inserted into a second living body lumen arranged side by side with the first living body lumen;
a puncture needle configured to puncture a living body tissue near the first insertion portion and the second insertion portion in a state where the first insertion portion is inserted in the first living body lumen and the second insertion portion is inserted in the second living body lumen;
a puncture needle guide portion which movably supports the puncture needle and which determines a puncture path for a needle tip of the puncture needle when the puncture needle punctures the living body tissue;
an ultrasonic transmission-reception section provided in the first insertion portion, the second insertion portion, or the puncture needle guide portion, for transmitting and receiving an ultrasonic wave; and
an image data production section for producing image data for displaying on a display section an image including the first living body lumen, the second living body lumen, and a predicted path of the puncture path, based on information acquired by the ultrasonic transmission-reception section.

10. The puncture apparatus according to claim 9, wherein the puncture needle punctures the living body tissue from a part different from the first living body lumen and the second living body lumen.

11. The puncture apparatus according to claim 9,
wherein the puncture needle has a bent region, and is rotatably disposed in the puncture needle guide portion; and
the puncture needle guide portion is configured to determine the puncture path such that the needle tip of the puncture needle passes between the first insertion portion and the second insertion portion when the puncture needle moves rotationally and punctures the living body tissue.

12. The puncture apparatus according to claim 9, comprising:
a puncture path changing section for changing the puncture path.

13. The puncture apparatus according to claim 12, wherein the puncture path changing section changes a position of the puncture needle relative to the insertion portion.

14. The puncture apparatus according to claim 13, comprising:
a detection section for detecting a positional relationship between the insertion portion and the puncture needle; and
wherein the image data production section is configured to determine the predicated path of the puncture path, based on results of detection by the detection section.

15. The puncture apparatus according to claim 12, wherein the predicted path of the puncture path is located between an urethra wall and a vaginal wall, and if the puncture path is not between the urethra wall and the vaginal wall, the puncture path is changed by the puncture path changing section so that the predicated path will be between the urethra wall and the vaginal wall.

16. A puncture apparatus comprising:
an elongated first insertion portion configured to be inserted into a first living body lumen, of the first living body lumen and a second living body lumen which are arranged side by side;
a puncture needle which punctures a living body tissue, with the first insertion portion inserted in the first living body lumen;
a puncture needle guide portion which rotatably supports the puncture needle, and determines a puncture path for a needle tip of the puncture needle such that the needle tip of the puncture needle passes a vicinity of the first insertion portion while avoiding the first insertion portion when the puncture needle punctures the living body tissue;
an ultrasonic transmission-reception section provided in the first insertion portion or the puncture needle guide portion and performs, for transmitting and receiving an ultrasonic wave; and
an image data production section for producing image data for displaying on a display section an image including the first living body lumen, the second living body lumen, and a predicted path of the puncture path, based on information acquired by the ultrasonic transmission-reception section.

17. The puncture apparatus according to claim 16, comprising:
an elongated second insertion portion configured to be inserted into the second living body lumen;
wherein the ultrasonic transmission-reception section is provided in the first insertion portion, the second insertion portion, or the puncture needle guide portion; and
the puncture needle punctures the living body tissue in a state in which the first insertion portion is inserted in the first living body lumen and the second insertion portion is inserted in the second living body lumen.

18. The puncture apparatus according to claim 16, wherein the puncture needle is configured to puncture the living body tissue from the second living body lumen.

19. The puncture apparatus according to claim 16, comprising:
a puncture path changing section for changing the puncture path, wherein the puncture path changing section changes a position of the puncture needle relative to the insertion portion;
a detection section for detecting a positional relationship between the insertion portion and the puncture needle; and
wherein the image data production section is configured to determine the predicated path of the puncture path, based on results of detection by the detection section.

20. The puncture apparatus according to claim 19, wherein the predicted path of the puncture path is located between an urethra wall and a vaginal wall, and if the puncture path is not between the urethra wall and the vaginal wall, the puncture path is changed by the puncture path changing section so that the predicated path will be between the urethra wall and the vaginal wall.
